(19) United States
(12) Patent Application Publication
(10) Pub. No.: US 2004/0208936 A1
(43) Pub. Date: Oct. 21, 2004

(54) Novel compositions

(76) Inventors: Roland Chorin, Mayenne (FR); James Cartwright, Mayenne (FR); Nathalie Barges Causeret, Mayenne (FR); Catherine Castan, Venissieux (FR); Florence Guimberteau, Venissieux (FR); Remi Meyrueix, Venissieux (FR)

Correspondence Address: GLAXOSMITHKLINE, Corporate Intellectual Property - UW2220, P.O. Box 1539, King of Prussia, PA, 19406-0939, US

(21) Appl. No.: 10/612,651

(22) Filed: Jul. 2, 2003

(30) Foreign Application Priority Data
Jul. 22, 2002 (GB) 0216993.6
Aug. 8, 2002 (GB) 0218423.2

(51) Int. Cl. A61K 9/16; A61K 9/50

(52) U.S. Cl. 424/490

(57) ABSTRACT

Modified release pharmaceutical formulations comprising microcapsules of amoxicillin, optionally immediate release amoxicillin and optionally clavulanate are of use in treating bacterial infections, in particular the empirical treatment of infections where penicillin resistant S pneumoniae may be implicated.

This invention relates to novel slow release granules of amoxicillin, their use in pharmaceutical formulations optionally further comprising potassium clavulanate and the use of these formulations in treating bacterial infections.

Amoxicillin is a well known β-lactam antibiotic widely used for treating bacterial infections. It is available in a wide range of pharmaceutical formulations, for instance swallow tablets, dispersible tablets, sachets, capsules and suspensions.

Potassium clavulanate is a known β-lactamase inhibitor. Products comprising amoxicillin and potassium clavulanate are marketed under the trade name “Augmentin” by GlaxoSmithKline. Such products are particularly effective for treatment of community acquired infections, in particular upper respiratory tract infections in adults and otitis media in children.

Various tablet formulations of amoxicillin and potassium clavulanate have been approved for marketing, comprising various different weights and ratios of amoxicillin and potassium clavulanate, for instance, conventional swallow tablets comprising 250/125, 500/125, 500/62.5, and 875/125 mg amoxicillin/clavulanic acid (in the form of potassium clavulanate). Such tablets comprise amoxicillin and clavulanic acid in the ratio 2:1, 4:1, 8:1 and 7:1, respectively. In addition, several such unit dosages are also available as single dose sachets, to provide an alternative for those who have difficulty in swallowing a tablet. A 1000/125 mg single dosage sachet is also available, in France.

Existing marketed tablet formulations of amoxicillin and amoxicillin/potassium clavulanate are conventional in that they provide immediate release of the active ingredients once the tablet reaches the stomach. There has also been some interest in developing formulations in which the release profile is modified, to allow for a longer interval between dosages, for instances, every 12 hours (bid, q12h), rather than every 8 hours (tid, q8h).

Thus, there have been various proposals to provide modified release formulations of amoxicillin, see for instance Arancibia et al (Int J of Clin Pharm, Ther and Tox, 1987, 25, 97-100), Hilton et al (International Journal of Pharmaceutics, 1992, 86, 79-88), Hilton et al (Journal of Pharmaceutical Sciences, 1993, 82, 737-743), Hoffman et al (Journal of Controlled Release, 1998, 54, 29-37 and WO 98/22091) and WO 00/61115 (Beecham Pharmaceuticals (PTE) Ltd)

Modified release formulations for amoxicillin and clavulanate have also been previously described in WO 94/27557 (SmithKline Beecham), WO 95/20946 (SmithKline Beecham), WO 95/28148 (SmithKline Beecham), WO 96/04908 (SmithKline Beecham), WO 00/61116 (Beecham Pharmaceuticals (PTE) Limited), WO 01/47499 (SmithKline Beecham plc), and WO 02/30392 (Beecham Pharmaceuticals (PTE) Limited). Of particular interest is that described in WO 00/61116 in which the preferred embodiment is a bilayer tablet comprising an immediate release layer having amoxicillin and potassium clavulanate and a slow release layer comprising sodium amoxicillin in combination with citric acid.

Of concern is the increasing resistance of pathogenic organisms, such as those found in respiratory tract infections, to anti-infective agents such as amoxicillin/potassium clavulanate, in particular drug resistant S pneumoniae. Increased resistance to penicillin of S pneumoniae (due to modified penicillin binding proteins) is developing around the world and is affecting clinical outcomes (see for instance Applebaum P C, Ped Inf Dis J, 1996, 15(10), 932-9). These penicillin resistant S pneumoniae (PRSP) have also been termed “DRSP” as they often exhibit decreased susceptibility not only to penicillin but also to a wider range of antimicrobial classes, including macrolides, azalides, beta-lactams, sulfonamides and tetracyclines. Amoxicillin (with or without clavulanate), along with some of the newer quinolones, has remained among the most active oral drugs against the increasingly resistant isolates of S pneumoniae, based on both MIC levels and pharmacokinetic properties of these compounds. Resistance rates (and MICs) have however continued to increase. Penicillin resistance in S. pneumoniae can be assessed according to criteria developed by the National Committee for Clinical Laboratory Standards (NCCLS), as follows: susceptible strains have MICs of ≦0.06 μg/ml, intermediate resistance is defined as an MIC in the range 0.12 to 1.0 μg/ml whilst penicillin resistance is defined as an MIC of ≧2 μg/ml. Furthermore, it is found that some 10% of pneumococci now have an amoxicillin MIC of 2 μg/ml.

There is consequently a need to provide new formulations of amoxicillin/clavulanate that combine the known safety profile and broad spectrum with improved activity against DRSP, including PRSP, with higher MICs, for the empiric treatment of respiratory infections where S pneumoniae, H influenzae and M catarrhalis are likely pathogens.

For β-lactams, including amoxicillin, it is recognised that the time above minimum inhibitory concentration (T>MIC) is the pharmacodynamic parameter most closely related to efficacy.

For a variety of β-lactams, a bacteriological cure rate of 85 to 100% is achieved when serum concentrations exceed the MIC for more than about 40% of the dosing interval (Craig and Andes, Ped Inf Dis J, 1996, 15, 255-259).

A further parameter which may be of importance is the ratio of the maximum plasma concentration (Cmax) to the MIC value, as this may be related to the potential to select for resistance. Too low a ratio may encourage the development of resistant strains. Preferably, the plasma Cₘₐₓ value is well above the MIC value, for instance, at least two times, more, preferably at least three times, most preferably at least four times, the MIC value.

To address this problem, a new suspension formulation comprising 600/43 mg/5 ml was made available by GlaxoSmithKline in the USA in 2001, as the product Augmentin ES 600, for use in treating children, at a dosage of 90/6.4 mg/kg day bid. This was initially described in WO 97/09042 (SmithKline Beecham), with clinical data subsequently presented by Bottenfield et al (Pediatr Infect Dis J, 1998, 17, 963-8).

In addition, GlaxoSmithKline are developing for adults a pharmacokinetically enhanced 1000/62.5 mg tablet formulation. This is a bilayer tablet having an immediate release layer comprising 563 mg of amoxicillin trihydrate and 62.5 mg of potassium clavulanate and a slow release layer comprising 438 mg of sodium amoxicillin and citric acid. It is believed that an intimate interaction between the sodium amoxicillin and citric acid leads to a modification in the release of amoxicillin. The tablet is used in a dosage regimen wherein the unit dosage is 2000/125 mg given every 12 h. There remains however a need to develop alternative formulations to provide the same unit dosage, for instance a single dose sachet for reconstitution into a suspension prior to administration, for those who might have difficulty in swallowing two tablets. This formulation was initially described in WO 00/61116 (Beecham Pharmaceuticals (PTE) Limited).

An alternative approach to providing formulations in which the release of drug substance is modified is Micropump® system developed by Flamel Technologies SA, Lyons, France and described in EP 0 709 087-A (Flamel Technologies SA). Small particles of drug substance are coated with a coating comprising four components, namely a film-forming polymer consisting of at least one non-hydrolysable cellulose derivative, a nitrogen-containing polymer, a plasticizer and a surface-active and/or lubricating agent, to form microcapsules. On administration, these microcapsules are held up in the upper part of the gastrointestinal tract. Drug substance is then slowly released from the microcapsules and absorbed through the walls of the GI tract. This technology has been applied to inter alia acetyl salicylic acid (aspirin) and aciclovir. Other exemplified drugs include captopril, atenolol and cimetidine. The exemplified coating composition comprises ethylcellulose (74%), polyvinylpyrrolidone (8%), castor oil(8%), and magnesium stearate (10%), applied from an acetone/iso-propanol solvent system. This approach can be contrasted with previous approaches which have relied on monolith tablets and then modifying the release by film coating the tablet or by forming a matrix from which drug is slowly released. These microcapsules are of reservoir variety, and can be contrasted with those of the matrix variety, such as those described in “Novel drug delivery and its therapeutic application” L. F. Prescott & W. S. Nimmo, Ed. John Wiley & Sons and by Duvemey et al, L'actualite chimique, December 1986.

A further modified release formulation system for amoxicillin and comprising at least three different dosage forms of differently coated granules is described in US 2001/0048944 A1 (Rudnic et al), a to a pulsed release profile. The different types of granules include granules with a pH-insensitive coating and granules with an enteric coating. A related application, US 2001/0046984 A1 (Rudnic et al), describes a modified release formulation for a β-lactam and a β-lactamase.

It has now been found that the approach described in EP 0 709 087-A (Flamel Technologies SA) may also be applied to amoxicillin, to allow the preparation of microcapsules having modified release properties.

Accordingly, in a first aspect, the present invention provides for microcapsules for use in a pharmaceutical formulation comprising microparticles of amoxicillin coated with at least one coating film comprising:

(a) at least one film-forming polymer (PI) present in a quantity of 50 to 90% by weight of dry matter of the whole coating composition, and comprising at least one water insoluble cellulose derivative;

(b) at least one nitrogen-containing polymer (P2), present in a quantity of 2 to 25% by weight of dry matter of the whole coating composition, and consisting of at least one polyacrylamide and/or one poly-N-vinylamide and/or one poly-N-vinyl-lactam;

(c) at least one plasticiser present in a quantity of 2 to 25% by weight of dry matter of the whole coating composition, selected from the group consisting of glycerol esters, phtalates, citrates, sebacates, cetylalcohol esters, castor oil and cutin; and

(d) optionally at least one surface-active and/or lubricating agent, present in a quantity of 2 to 20% by weight of dry matter of the whole coating composition, and selected from one or more of anionic surfactants, and/or from nonionic surfactants, and/or from lubricants selected from stearates, stearylfumarates, lauryl sulfates and/or glyceryl behenate;

said microparticles of amoxicillin having a particle size of between 50 and 1000 μm; and

said coating being present in from 5 to 45% by weight of the granule.

As used herein, the term “microcapsule” refers to the film-coated particles whilst the term “microparticles” refers to the particles of non-film-coated amoxicillin.

The microcapsules of the present invention are designed in such a way as to retard their transit through the small intestine, so as to remain in the upper part of the GI tract for a period of at least about 4 hours, preferably of at least about 6 hours thereby permitting the absorption of the amoxicillin during at least part of the residence time thereof in the small intestine.

In addition, microcapsules according to the present invention are less prone to cause irritancy in the GI tract, even in the case of prolonged adhesion to the GI mucous membranes, because each microcapsule contains only a fraction of the total amoxicillin dosage. The small particle size of the microcapsules also allows for great uniformity of transit.

Preferably, the film-forming polymer (PI) is present in a quantity of 50 to 80%, more preferably 60 to 80%, by weight of dry matter of the whole coating composition. Preferably, the film-forming polymer (PI) is ethylcellulose and/or cellulose acetate, more preferably ethyl cellulose. A representative grade of ethyl cellulose is the product Ethocel Std 7 Premium, available from Dow Chemical, which has a solution viscosity of 6 to 8 mPa s and a mean particle size of 210 μm (Handbook of Pharmacutical Excipients Third Edition, 2000, American Pharmaceutical Association and Pharmaceutical Press, 195) and conforming to Phar Eur 2001, 1999, no. 822.

Ethyl cellulose may be used for the coating process dissolved in an organic solvent or solvent mixture. Ethyl cellulose may also be used as an aqueous polymer dispersion (Ethylcellulose Aqueous Dispersion USP 25 NF20, page 2546), for instance an Aquacoat product available from FMC Corp, in particular the product Aquacoat ECD30 (30% aqueous dispersion). This comprises ethyl cellulose (27%) and, to assist in the formation and stabilisation of the dispersion, cetyl alcohol (2.5%) and sodium lauryl sulfate (1.3%), by weight.

Preferably, the nitrogen-containing polymer (P2) is present in a quantity of 2 to 20%, more preferably 10 to 20%, by weight of dry matter of the whole coating composition. Preferably, the nitrogen-containing polymer (P2) is a polyacrylamide and/or a polyvinyl lactam, for instance a polyvinylpyrrolidone, more preferably polyvinylpyrrolidone (PVP). A representative grade of polyvinylpyrrolidone is as described in USP 24 NF19, page 1372, for example the product Plasdone K29/32 (available from ISP).

Preferably, the plasticiser is present in a quantity of 4 to 15%, more preferably 5 to 10% by weight of dry matter of the whole coating composition. Preferably, the plasticiser is dibutyl sebacate, triethyl citrate or castor oil, more preferably castor oil, for use with ethyl cellulose in an organic or aqueous alcohol solvent system solvent. Preferably, the plasticiser is a citrate such as triethyl citrate or a sebacate such as dibutyl sebacate, for use with an aqueous dispersion of ethylcellulose.

The coating composition preferably includes a surface-active and/or lubricating agent. This component is may be incorporated to assists the coating process, by reducing an initial tendency for the microparticles to agglomerate. The surface-active and/or lubricating agent is preferably present in a quantity of 2 to 15%, more preferably 2 to 10%, still more preferably 2 to 8%, by weight of dry matter of the whole coating composition. Preferred anionic surfactants include the alkali metal or alkakine-earth metal salts of fatty acids, in particular stearic acid and/or oleic acid and C₍₁₂₋₂₀₎alkyl sulfates, for instance sodium lauryl sulfate. Preferred nonionic surfactants include polyoxyethylene alkyl ethers, polyoxyethylenated esters of sorbitan, polyoxyethylenated derivatives of castor oil and C₍₁₂₋₂₀₎alcohols such as cetyl alcohol. Preferred lubricants include stearates, in particular calcium, magnesium, aluminium or zinc stearate, stearylfumarates, in particular sodium stearylfumarate and/or glyceryl behenate. Preferably, the surface-active is magnesium stearate or a polyoxyethylenated derivative of castor oil, for instance polyoxyl 40 hydrogenated castor oil, more preferably polyoxyl 40 hydrogenated castor oil, for example the product Cremophor RH40 (BASF).

A representative coating composition comprises ethylcellulose present in from 60 to 80%, polyvinylpyrrolidone present in from 10 to 20%, castor oil present in from 5 to 10%, and polyoxyl 40 hydrogenated castor oil present in from 2 to 8%, by weight of the of dry matter of the whole coating composition. A typical coating comprises ethyl cellulose (about 70 to 72%), polvinylpyrrolidone (about 16 to 18%), castor oil (about 8%) and polyoxyl 40 hydrogenated castor oil (about 4%), ±5% of each component, by weight of the of dry matter of the whole coating composition.

A further representative coating composition is prepared from an aqueous dispersion of ethyl cellulose and comprises ethyl cellulose present in from 60 to 80%, cetyl alcohol present in from 4 to 10%, sodium lauryl sulfate present in from 2 to 5%, polyvinylpyrrolidone present in from 2 to 10%, and dibutyl sebacate present in from 10 to 25%, respectively, by weight of the of dry matter of the whole coating composition. A typical coating composition prepared from an aqueous dispersion of ethyl cellulose comprises ethyl cellulose (about 76%), polyvinylpyrrolidone (about 3%), and dibutyl sebacate or triethyl citrate (about 21%), respectively, ±5% of each component, by weight of the three components. A further typical coating composition prepared from an aqueous dispersion of ethyl cellulose comprises ethyl cellulose (about 84%), polyvinylpyrrolidone (about 3%), and dibutyl sebacate or triethyl citrate (about 13%), respectively, ±5% of each component, by weight of the three components.

The coating composition may further comprise additional adjuvants conventionally used in the field of film forming, added to assist dispersion of the coating composition in an aqueous solvent system, pigments and fillers.

Preferably, the coating represents from 5 to 45%, more preferably from 7 to 20%, advantageously from 9 to 15%, more advantageously from 10 to 15%, of the weight of the microcapsules. It is found that the level of coating is a key element determining the release characteristics of the microcapsules of the present invention.

Preferably, the film coating has a thickness of between 2 and 100 μm, more preferably between 5 and 25 μm. Such coating films are found to have sufficient mechanical strength to prevent their breaking and/or splitting in the GI tract, up to and beyond completion of the release of amoxicillin from the microcapsule.

Microcapsules of the present invention may be further coated with at least one additional coating layer, to further modify the release profile thereof. Thus, microcapsules may be further coated with, for example, an enteric coating layer, for instance a methacrylic acid copolymer such as a Eudragit L 100 (Rohm and Haas). This may be used in combination with a plasticizer conventionally used with such, for instance, triethyl citrate. Typically, the enteric coating is applied at a loading of from 10 to 40% by weight of the microcapsule.

Preferably, the particle size of the amoxicillin microparticles is between 100 and 750 μm, more preferably, between 100 and 500 μm, advantageously between 200 and 400 μm, more advantageously between 250 and 400 μm. Microparticles of the appropriate size may be obtained by sieving bulk material through appropriate sieves, to remove both larger and smaller sized particles. It is found that the dissolution profile is very sensitive to the size of the microparticles. In addition, larger particles are avoided as they tend to have an adverse effect on palatability.

The microcapsules preferably comprise amoxicillin in an amount of between 55 and 95%, more preferably between 80 and 93%, advantageously between 85 and 90%, by weight of the microcapsule.

The amoxicillin for use in preparing microcapsules of the present invention may be substantially pure amoxicillin or it may be have been subjected to a preliminary processing step, to form granulates. Such a processing step may be a simple granulation step, for instance a conventional wet granulation process (see EP 0 281 200, Gist Brocades NV), or a dry compaction process such as roller compaction, to provide particles with more consistent properties for microcapsule formation.

Preferred granulates are prepared by roller compaction and may also include processing aids conventionally used in such granulates, for instance an intragranular lubricant such as magnesium stearate, used at a low level, for instance from 0.5 to 2% by weight of amoxicillin. Suitable amoxicillin granulates for use in formulations of the present invention have been previously described in WO 98/35672 (SmithKline Beecham Laboratoires Pharmaceutique), WO 92/19277 (SmithKline Beecham) and GB 2 005 538-A (Beecham Group). Such granulates may further comprise an intragranular disintegrant such as maize starch and rice starch, crospovidone (cross-linked N-vinyl-2-pyrrolidinone, CLPVP), sodium starch glycollate, croscarmellose sodium and formaldehyde-casein, or combinations thereof, present in from 0.1 to 10%, preferably from 1.0 to 8.0%, more preferably from 1.25 to 3.5% by weight of the granulate. Such amoxicillin granulates may be prepared by roller compaction of the amoxicillin and intra-granular ingredients, followed by milling and sizing, as described in WO 98/35672 (SmithKline Beecham Laboratoires Pharmaceutique).

The microcapsules of the present invention may be admixed with at least one anti-agglomerating agent formed, for instance, talc, colloidal silica or of a mixture of the two, in an amount, for example, of from 0.5 to 5% by weight, preferably of from 1.5 to 3% by weight. This is found to be of assistance in reducing the degree of caking of the microcapsules.

The release of amoxicillin from the microcapsules of the present invention is a diffusion controlled process. The in vitro dissolution properties of microcapsules of the present invention may be readily determined using standard dissolution techniques, for instance a dissolution method described in the USP. Microcapsules of the present invention generally have a first order (quasi-exponential) release profile. Microcapsules which have been further coated with an enteric coating are found to have a sigmoidal release profile, in which there is an initial delay time, before the microcapsules reach the part of the GI tract at which the enteric coating will start to dissolve.

In a first release profile, microcapsules release between 10 and 30% amoxicillin after 0.5 h, between 40 and 60% after 2 h and at least 70% after 6 h (measured at pH 6.8). A typical target is for about 50% release by between 1.5 and 2 h, typically about 1.7 hours. In a second release profile, microcapsules release between 15 and 40% amoxicillin after 0.5 h, between 60 and 90% after 2 h and at least 90% after 6 h (measured at pH 6.8). A typical target for this is for about 70% release between 1.8 and 2.2 h, typically by about 2 hours. It is found that the release profile is sensitive to the thickness of the coating and the size distribution of the uncoated microparticles of amoxicillin.

It is further found that the release profiles of microcapsules according to the present invention may be substantially maintained after up to ten days storage as a suspension in water, at 5° C., in particular when dibutyl sebacate is used as the plasticiser.

In general, microcapsules of amoxicillin according to the present invention may be prepared by spray coating microparticles of amoxicillin with the coating film, as a dispersion or a suspension in a solvent system, as described in EP 0 709 087 (Flamel Technologies SA).

Accordingly, in a further aspect, the present invention provides for a process for preparing microcapsules of amoxicillin which process comprises applying in a film coating apparatus a coating composition prepared by admixing the components of the film coating in a solvent system to microparticles of amoxicillin and thereafter drying the microcapsules thus obtained, and optionally, and if necessary, admixing these microcapsules with at least one anti-agglomerating agent.

Solvents suitable for use in the present process include organic solvents, for example, ketones, esters, chlorinated solvents, and alcohols, preferably those comprising C₍₁₋₆₎alkyl, such as, acetone, methyl ethyl ketone, methanol, ethanol, iso-propanol, cyclohexane and methylene chloride, and mixtures thereof, in particular acetone/iso-propanol.

For amoxicillin, it is found that there is a tendency for electrostatic to develop, when using certain organic solvents. It was however found that this could be avoided by using an aqueous alcohol solvent system comprising an alcohol, such as ethanol, and water, for instance a mixture of ethanol and water in the ratio between 60:40 and 80:20, typically about 70:30, in preference to mixed organic solvent systems such as acetone/iso-propanol previously favoured.

Accordingly, in a further aspect, the present invention provides a process which process comprises applying a coating composition prepared by admixing the components of the film coating in an aqueous alcohol solvent system to microparticles of amoxicillin and thereafter drying the microcapsules thus obtained, and optionally, and if necessary, mixing these microcapsules with at least one anti-agglomerating agent.

Typically, a coating composition for use with an aqueous alcohol solvent system comprises ethylcellulose, polyvinylpyrrolidone, castor oil, and polyoxyl 40 hydrogenated castor oil, present in from 60 to 80%, 10 to 20%, 5 to 10%, and 2 to 8%, respectively, by weight of the total of the components of the coating.

Typically, the concentration of the film forming components is in the range 5 to 15% by weight of the solution.

Furthermore, it was also found that aqueous solvents could be advantageously used, for instance 100% water. Such a solvent system may also require a further or curing step, after the coating step, to ensure that the ethyl cellulose latex coalesces, to form a continuous and stable film. Curing is preferably carried out at a temperature above the glass transition temperature of ethyl cellulose, for instance about 60° C., preferably for at least 0.5 h, on a tray in a ventilated or a non-ventilated oven or in a fluidised bed, according to conventional methodolgy.

Accordingly, in a further aspect, the present invention provides a which process comprises applying a coating composition prepared by admixing the components of the film coating in an aqueous solvent system to microparticles of amoxicillin and thereafter curing the microcapsules thus obtained, and optionally, and if necessary, admixing these microcapsules with at least one anti-agglomerating agent.

Typically, a coating composition for use with an aqueous solvent system comprises an aqueous dispersion comprising ethyl cellulose and comprises ethyl cellulose present in from 60 to 80%, cetyl alcohol present in from 4 to 10%, sodium lauryl sulfate present in from 2 to 5%, polyvinylpyrrolidone present in from 2 to 10%, and dibutyl sebacate present in from 10 to 25%, respectively, by weight of the of dry matter of the whole coating composition.

Typically, the film forming components are present in from 60 to 80, typically about 70% by weight of the aqueous dispersion.

In a further aspect, the present invention provides for microcapsules of amoxicillin obtainable by applying a coating composition prepared by admixing the components of the film coating in an aqueous solvent system to microparticles of amoxicillin and thereafter curing the microcapsules thus obtained.

Such microparticles are found to have an enhanced pharmacokinetic profile.

The coating composition/solvent system mixture may be applied by spraying onto the microparticles of amoxicillin which are set in motion, preferably by mechanical stirring or more preferably, by fluidization. Suitable equipment is available commercially, for instance Glatt

In a preferred embodiment of the process, microcapsules of amoxicillin are prepared according to a process which comprises the following steps:

(a) preparing a mixture comprising ethyl cellulose castor oil, and polyvinylpyrrolidone in solution, in a water/ethanol mixture such that the water/ethanol volume ratio is between 40/60 and 20/80;

(b) adding surfactant to the solution of (a) to form a suspension;

(c) spraying the microparticles of amoxicillin with the resultant mixture in a fluidized bed,

(d) drying the microcapsules from (c) in a fluidized bed and/or oven, and

(e) thereafter and if necessary, admixing the microcapsules from (d) with 0.5 to 3% by weight of anti-adhesion agent, on the basis of 100% of final product obtained after mixing.

In a further preferred embodiment of the process, microcapsules of amoxicillin are prepared according to a process which comprises the following steps:

(a) preparing a mixture comprising from ethyl cellulose, introduced in the form of an aqueous dispersion, a plasticizer, and polyvinylpyrrolidone in solution, in water;

(b) spraying the microparticles of amoxicillin with the resultant mixture in a fluidized bed,

(c) drying the microcapsules from (c) in a fluidized bed and/or oven, and

(d) thereafter and if necessary, admixing the microcapsules from (d) with 0.5 to 3% by weight of anti-adhesion agent, on the basis of 100% of final product obtained after mixing.

A further, enteric coating may be applied, if so desired to microcapsules by spray coating a coating composition such as Eudragit L 100 and triethyl citrate in an organic solvent system, for instance acetone/water, according to conventional means.

The amoxicillin microcapsules herein before described may be incorporated into a pharmaceutical formulation.

Accordingly, in a further aspect, the present invention provides for a modified release pharmaceutical formulation which comprises amoxicillin, and optionally potassium clavulanate such that the ratio of amoxicillin to clavulanate (if present) is from 2:1 to 30:1, and in which from 30 to 100% of the amoxicillin content is present as microcapsules of amoxicillin, to provide a slow release phase and from 0 to 70% of the amoxicillin content is present in an immediate release form, to provide an immediate release phase, further admixed with pharmaceutically acceptable excipients.

Preferably the ratio of amoxicillin to clavulanate is from 2:1 to 20:1, more preferably 6:1 to 20:1, more preferably 12:1 to 20:1, most preferably 14:1 to 16:1. Typical ratios include 2:1, 4:1, 7:1, 8:1, 14:1, 15:1, and 16:1.

Preferably, from 40 to 100%, more preferably from 50 to 90%, of the amoxicillin content is present as microcapsules of amoxicillin. Representative amounts include 60, 65, 70, 80 and 87.5%. The formulation may comprise microcapsules which all have substantially the same release profile or it may comprise at least two types of microcapsules which have differing release profiles.

It is found useful to include some immediate release amoxicillin, to ensure an adequate Cₘₐₓ value.

As used herein, the term “modified release” refers to a release of drug substance from a pharmaceutical formulation which is at a slower rate than from an immediate release formulation such as a conventional swallow tablet or capsule and may include an immediate release phase and a slow release phase and/or a delayed slow release phase. Modified release formulations are well known in the art, see for instance Remington: The Science and Practice of Pharmacy, Nineteenth Edn, 1995, Mack Publishing Co, Pennsylvania, USA.

As used herein, the term “immediate release” refers to the release of the majority of the active material content within a relatively short time, for example within 1 hour, preferably within 30 minutes, after oral ingestion.

As used herein, the term “slow release” refers to the gradual but continuous or sustained release over a relatively extended period of the active material content (in this case amoxicillin) after oral ingestion and which starts when the formulation reaches the stomach and amoxicillin starts to diffuse through the coating layer. The release will continue over a period of time and may continue through until and after the formulation reaches the intestine. As used herein, the term “delayed slow release” refers to a release profile in which there is an initial delay in release because the microcapsules have an enteric coating so that the slow release of amoxicillin does not start until the formulation reaches the intestine when the increasing pH causes the enteric coating to dissolve. This can be contrasted with the term “delayed release” in which release of the active does not start immediately the formulation reaches the stomach but is delayed for a period of time, for instance until when the formulation reaches the intestine when the increasing pH is used to trigger release of the active from the formulation.

Preferably, the modified release formulations of the present invention are formulated such that the release of amoxicillin is effected predominantly through the stomach and small intestine, so that absorption through the specific amoxicillin absorption site in the small intestine is maximised. Preferably, the amoxicillin release profile is made up of a contribution from an immediate release component which is then complemented and extended by an on-going contribution from a slow release component. Preferably, potassium clavulanate, if present, is released substantially immediately from the formulation, when the formulation reaches the stomach and is absorbed therefrom, thereby minimising the risk of degradation from prolonged exposure to the stomach. Such formulations are preferably formulated such that the release of amoxicillin and potassium clavulanate occurs predominantly within 3 hours of ingestion of the formulation.

Preferably, the modified release formulation has an in vitro dissolution profile in which 25 to 70%, preferably 35 to 70% of the amoxicillin content is dissolved within 30 min; further in which 40 to 75%, preferably 45 to 65% of the amoxicillin content is dissolved within 60 min; further in which 50 to 85%, preferably 60 to 70% of the amoxicillin content is dissolved within 120 min; further in which 60 to 95%, preferably 70 to 85% of the amoxicillin content is dissolved within 180 min; and further in which 70 to 100%, preferably 75 to 100% of the amoxicillin content is dissolved within 240 min. In comparison, a conventional, immediate release amoxicillin tablet dissolves essentially completely within 30 minutes. The dissolution profile may be measured in a standard dissolution assay, as hereinbefore described for the constituent microcapsules.

It will be appreciated that a modified release formulation of the present invention will have a profile in vivo with respect to amoxicillin, which is made up of an of an initial burst from the immediate release phase (if present) to provide an acceptable Cₘₐₓ value, supplemented by a further contribution from the slow release phase, to extend the T>MIC parameter to an acceptable value.

Preferably, the modified formulation provides an “Area Under the Curve” (AUC) value which is substantially similar to, for instance at least 80%, preferably at least 90%, of that of the corresponding dosage of amoxicillin taken as a conventional (immediate release) formulation, over the same dosage period, thereby maximising the absorption of the amoxicillin component from the slow release component.

The pharmacokinetic profile for a dosage of the present invention may be readily determined from a single dosage bioavailability study in human volunteers. Plasma concentrations of amoxicillin may then be readily determined in blood samples taken from patients according to procedures well known and documented in the art.

Amoxicillin for use in the immediate release phase may be optionally provided as granulates which may optionally further comprise an intragranular lubricant such as magnesium stearate and optionally an intragranular disintegrant such as CLPVP, as hereinbefore described. The terms “amoxicillin” and “clavulanate” used herein, and unless otherwise specified, include both the free parent acids and derivatives such as salts thereof. Weights and ratios are expressed in terms of the weight of parent compound amoxicillin or clavulanic acid, this terminology being used throughout this description unless otherwise stated. In addition, it will be appreciated that in practice, weights of amoxicillin and clavulanate to be incorporated into a formulation will be further adjusted, in accord with conventional practice, to take account of the potency of the amoxicillin and clavulanate.

Suitable derivatives of amoxicillin are amoxicillin trihydrate, anhydrous amoxicillin and alkali metal salts of amoxicillin such as sodium amoxicillin. Preferably amoxicillin is used as amoxicillin trihydrate. Preferably, the equilibrium relative humidity (ERH) of the amoxicillin trihydrate is carefully controlled by appropriate drying so that it doe not compromise other aspects of the formulation, in particular clavulanate stability. Preferably, the ERH is less than 50%, more preferably less than 30%, most preferably from 10 to 20%.

Suitable derivatives of clavulanic acid are alkali metal salts of clavulanic acid such as potassium clavulanate. Potassium clavulanate is conventionally incorporated into a formulation in combination with a diluent such as silica gel, for instance the product Syloid AL-1, which will also provide a desiccating function, or microcrystalline cellulose, typically in a ratio of 1:1.

At least part of the amoxicillin may be provided as granulates further comprising potassium clavulanate, for instance as granulate comprising amoxicillin and clavulanate in the ratio 2:1, with further amoxicillin to make up the total amount provided as amoxicillin granulates. Alternatively, all the amoxicillin and potassium clavulanate for use in the immediate release phase and some or all of the remaining excipients may be processed together in a roller compactor and then milled and sieved to provide immediate release phase granulates. Preferably such granulates will have a similar size to the microcapsules, to provide a homogenous formulation. Granulates comprising amoxicillin/clavulanate have been previously described in WO 98/35672 (SmithKline Beecham Laboratoires Pharmaceutique).

Preferably, formulations of this invention comprise amoxicillin trihydrate and potassium clavulanate, this combination having met with regulatory approval, and being particularly advantageous.

The pharmaceutical formulations of the present invention may be provided in a variety of different presentations, for instance as a single dose sachet, as capsules, a dry powder for reconstitution to provide a multiple dosage suspension, or as tablets.

When in the form of a sachet, the contents will be free-flowing, so that the contents of the sachet may be readily added to water immediately prior to use, to form a suspension which is then consumed. Typically, a single sachet will comprise the unit dosage. This may be a unit dosage that is appropriate for an adult patient. A smaller unit dosage may also be provided, for younger patients. The sachet formulation may comprise further excipients such as silica gel, sweeteners, and flavours. Such sachets may preferably be provided as ‘sugar-free’ formulations, comprising, an artificial sweetener such as aspartame, rather than sugar. Preferably, such formulations comprise from 1 to 10%, more preferably from 1 to 5% aspartame. Other excipients which may be incorporated include thickeners, preservatives such as sodium benzoate and buffers such as sodium citrate.

Further suitable formulations for amoxicillin include encapsulated formulations which may optionally include a lubricant, present in an amount of less than 0.5% by weight of the granulates, and being contained within a pharmaceutical capsule. The pharmaceutical capsule may be an entirely conventional capsule, capable of dissolving in the stomach to release its contents, for example made of gelatine.

Sachets may comprise from 250 to 3000 mg of amoxicillin, for instance 250, 500, 600, 800, 1000, 1500, 2000, 2500, 3000 mg amoxicillin. Representative sachets comprise 500, 600, 800, 1000 and 2000 mg.

Suitable tablet formulation include dispersible tablets. For large tablets, for instance tablets comprising 1000 mg amoxicillin, a preferred type is a rapidly-dispersible tablet which may be taken orally and allowed to disperse in the mouth or, more preferably, dispersed in water immediately prior to taking, and then swallowed as a liquid. Dispersible tablets typically comprise a disintegrating agent, for instance cellulose-based products such microfine cellulose, microcrystalline cellulose or hydroxypropyl cellulose as well as sodium starch glycollate and CLPVP. The disintegrant is preferably present in from 5 to 30%, more preferably from 5 to 15%, based on the weight of the final tablet. Dispersible tablets also typically contain conventional lubricants such as magnesium stearate, sweetening agents such as aspartame or sodium saccharin and flavouring and colouring agents.

A further suitable tablet formulation is a chewable tablet with further compromises excipients conventionally used to form a chewable base, for instance, mannitol, sorbitol, dextrose, fructose, or lactose, alone or in combination, preferably present in from 10 to 30% based on the weight of the final tablet, more preferably from 15 to 25%. The tablets may also contain conventional lubricants such as magnesium stearate, sweetening agents such as aspartame or sodium saccharin and flavouring and colouring agents.

Formulations such as capsules and tablets will typically comprise from 250 to 1100 mg of amoxicillin, for instance 250, 500, 875 and 1000 mg amoxicillin. A unit dosage may be provided by a single tablet or capsule, or as two or tablets or capsules, for larger (greater than 1000 mg) unit dosages.

Typically, the amount of clavulanate in a formulation will be adapted such that dosage will provide 125 mg of potassium clavulanate, the amount approved in existing regimens, for adults. A representative sachet according to the present invention comprises 2000/125 mg amoxicillin/clavulanate, corresponding to two tablets of 1000/62.5 mg, as described in WO 00/61116 (Beecham Pharmaceuticals (PTE) Limited). A representative tablet according to the present invention comprises 1000/62.5 mg amoxicillin/clavulanate.

Other suitable formulations include dry powder formulations for paediatric use which are reconstituted into aqueous suspensions prior to use, to provide a multiple dose suspension. Such formulations may comprise further excipients such as, for example, a desiccating agent, for instance silica gel or dried maltodextrin, thickeners such as xanthan gum, carboxymethylcellulose sodium, silica gel, and hydroxypropylmethyl cellulose, preservatives such as sodium benzoate, sweetening agents such as aspartame, acesulfamate potassium, and sodium saccharin, lubricants such as magnesium stearate, glidants such as colloidal silicon dioxide, and flavouring agents such as fruit flavour(s). The presence of an immediate release amoxicillin component is found to assist the maintenance of the in vitro release profile of the microcapsules.

Formulations of amoxicillin and optionally clavulanate are provided comprising from 200 to 1200 mg of amoxicillin per unit amount of formulation and optionally from 25 to 80 mg of clavulanate per unit amount of formulation, such that the ratio of amoxicillin to clavulanate is in the range 2:1 to 30:1. Preferably, formulations are provided comprising from 400 to 1200 mg of amoxicillin per unit amount of formulation, for instance 400, 500, 600, 800 and 1000 mg/per unit amount of formulation, typically 600 and 800 mg/per unit amount of formulation and optionally from 40 to 80 mg of clavulanate per unit amount of formulation. As used herein, the term “amount of formulation” refers to the amount of the dry formulation which is reconstituted to provide 5 ml of reconstituted suspension and therefore corresponds to the concentration in 5 ml.

Formulations according to the present invention are of use in treating bacterial infections in humans. Accordingly, in a further aspect, the present invention provides for the use of amoxicillin and optionally clavulanate, as hereinbefore defined, in the manufacture of a medicament for treating bacterial infections, preferably at intervals of about 12 h.

In a preferred aspect, the present invention provides for the use of 2000 mg of amoxicillin and optionally 125 mg clavulanate in the manufacture of a medicament, as hereinbefore defined, for treating bacterial infections, in particular in adult patients, at intervals of about 12 h.

In a further aspect, the present invention provides for a mean plasma concentration of amoxicillin of 4 μg/mL for at least 4.6 h, preferably at least 4.8 h, more preferably at least 5.5 h, typically between 5.5 and 6.5 h.

In a further aspect, the present invention provides for a mean plasma concentration of amoxicillin of 8 μg/ml for at least 4.2 h, preferably at least 4.4 h, more preferably at least 4.8 h.

In a further aspect, the present invention provides for a mean maximum plasma concentration (Cₘₐₓ) of amoxicillin which is at least 12 μg/mL, preferably at least 14 μg/mL, most preferably at least 16 μg/mL, typically from 16 to 20 μg/mL.

Preferably, the mean plasma concentration of amoxicillin and the mean maximum plasma concentration of amoxicillin are measured after oral administration of a formulation comprising amoxicillin at the start of a light meal.

Preferably, a formulation of the invention, for instance a sachet or a tablet formulation, is used in a method of treating bacterial infection in an adult which comprises administering to a patient in need thereof a dosage of from 1900 to 2600 mg, typically 2000 mg of amoxicillin, and optionally 125 mg of clavulanate, every 12 hours.

In a further aspect, the present invention provides for a method of treating bacterial infection in a paediatric patient which method comprises administering to a patient in need thereof a dosage of from 80 to 180 mg/kg/day of amoxicillin, preferably from 120 to 160, more preferably about 150 mg/kg/day and, optionally, from 6 to 11 mg/kg/day, in divided dosages every 12 h, from a modified release formulation.

In a further aspect, the present invention provides for a method of treating bacterial infection in a paediatric patient which method comprises administering to a patient in need thereof a dosage of from 80 to 180 mg/kg/day of amoxicillin, preferably from 120 to 160, more preferably about 150 mg/kg/day and, optionally, from 6 to 7 mg/kg/day, preferably from 6.3 to 6.7 mg/kg/day of clavulanate, in divided dosages every 12 h, from a modified release formulation.

In a further aspect, the present invention provides for a method of treating bacterial infection in a paediatric patient which method comprises administering to a patient in need thereof a dosage of from 80 to 180 mg/kg/day of amoxicillin, preferably from 120 to 160, more preferably about 150 mg/kg/day and, optionally, from 9 to 11 mg/kg/day, preferably about 10 mg/kg/day of clavulanate, in divided dosages every 12 h, from a modified release formulation.

Preferably, the modified release formulation for use in the method for treating a paediatric patient provides a mean plasma concentration of amoxicillin of 8 μg/ml for at least 4.2 h, preferably at least 4.4 h, more preferably at least 4.8 h.

Preferably, the method for treating a paediatric patient uses a formulation of the present invention, for instance a dry powder formulation which is reconstituted into an aqueous suspension.

Bacterial infections amenable to the present invention include infections caused by the organisms S pneumoniae (including Drug Resistant S pneumoniae (DRSP), for instance Penicillin Resistant S pneumoniae (PRSP)), and/or the β-lactamase producing respiratory pathogens, most notably H influenzae and M catarrhalis, such as respiratory tract infections, including community acquired pneumoniae (CAP), acute exacerbations of chronic bronchitis (AECB) and acute bacterial sinusitis (ABS), and, for paediatric patients, acute otitis media. The higher break points achievable through the improved pharmacokinetic profile will be especially advantageous compared to existing antibacterial agents. Most outpatient respiratory infections are caused by either S pneumoniae and/or the β-lactamase producing bacteria and are treated empirically so there is a continuing need for a method of treatment, such as the present invention, that provides a spectrum of activity that covers all such pathogens. The duration of therapy will generally between 5 and 14 days, typically 5 or 7 days for indications such as acute exacerbations of chronic bronchitis but 10 days for acute bacterial sinusitis.

Further bacterial infections amenable to the present invention wherein the formulation is amoxicillin only and has no clavulanate include infections caused by the organism Streptococcus pyogenes, for instance acute bacterial tonsillitis and/or pharyngitis. The duration of therapy will generally between 7 and 14 days, typically 7 days for most indications but 10 days for acute bacterial sinusitis.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

This invention will now be illustrated by reference to the following examples.

### EXAMPLES

### Examples 1-7

### Microcapsules M1 to M7 Comprising Amoxicillin

### Example 1

### Microcapsules M1

| | Q (g) |
|---|---|
| Ingredient | |
| Amoxicillin/CLPVP 200-500 μm granulate | 700.0 |
| Ethocel 7 premium¹ | 68.7 |
| Plasdone K29/32² | 15.3 |
| Castor oil | 7.6 |
| Cremophor RH 40³ | 3.8 |
| Total | 795.4 |
| Solvents | |
| Ethanol 100% | 601.4 |
| Water | 257.7 |

¹ethyl cellulose
²polyvinylpyrrolidone
³polyoxyl 40 hydrogenated castor oil
First order release profile (50% release time of 1.7 hours at neutral pH). Coating level 12%

Preparation of Microcapsules

Amoxicillin/polyvinylpyrrolidone granulates (prepared according to the process described in WO98/35672 (SmithKline Beecham Laboratoires Pharmaceutique)) were sieved to separate out microparticles having a size between 200 and 500 μm.

The coating composition was prepared by adding the components to water/ethanol (70:30) and stirring over a period of several hours until a uniform solution was formed.

The microparticles (700 g) were coated by spray drying with the coating composition in a fluid bed coater (Glatt Pharmatech). The typical yield obtained is greater than 95%.

### Example 2

### Microcapsules M2

| | Q (g) |
|---|---|
| Ingredient | |
| Amoxicillin/CLPVP 200-400 μm granulate | 1000.0 |
| Ethocel 7 premium | 84.5 |
| Plasdone K29/32 | 18.8 |
| Castor oil | 9.4 |
| Cremophor RH 40 | 4.7 |
| Total | 1117.4 |
| Solvents | |
| Ethanol 100% | 884.0 |
| Water | 378.9 |

Coating level = 10.5%
First order release profile (70% release time of 2 hours at neutral pH).

### Example 3

### Microcapsules M3

| | Q (g) |
|---|---|
| Ingredient | |
| Amoxicillin/CLPVP 200-400 μm granulate | 1000.0 |
| Ethocel 7 premium | 127.1 |
| Plasdone K29/32 | 28.2 |
| Castor oil | 14.1 |
| Cremophor RH 40 | 7.1 |
| Total | 1176.5 |
| Solvents | |
| Ethanol 100% | 1111.8 |
| Water | 476.5 |

First order release profile (50% release time of 1.7 hours at neutral pH)
Coating level = 15%

### Example 4

### Microcapsules M4 (Aqueous Solvent System)

| | Q (g) |
|---|---|
| Ingredient | |
| Amoxicillin 200-400 μm granule | 1000.0 |
| Aquacoat ECD30¹ | 290.6 |
| Dibutyl sebacate | 24.1 |
| Plasdone K29/32 | 3.8 |
| Total | 1318.5 |
| Solvents | |
| Water | 150.8 |

¹30% by weight aqueous dispersion of ethylcellulose polymer (27%) further comprising cetyl alcohol (2.5%) and sodium lauryl sulfate (1.3%) (by weight)

The coating composition was prepared by adding the components to water and stirring over a period of several hours until a homogeneous dispersion was formed.

The microparticles (1000 g) were coated by spray drying with the coating composition in a fluid/bed coater (Glatt Pharmatech). The typical yield obtained is greater than 95%.

For microcapsules prepared from an aqueous dispersion of ethylcellulose, a further curing step is required, after the coating step, to ensure that the ethyl cellulose latex coalesces, to form a continuous and stable film. Curing is effected at 60° C., for 4 h, in a ventialted or a non-ventilated oven.

Coating level=10.5%

First order release profile (50% release time of 1.7 hours at neutral pH)

### Example 5

### Microcapsules M5

| | Q (g) |
|---|---|
| Ingredient | |
| Amoxicillin 200-400 μm granule | 700.0 |
| Ethocel 7 premium | 93.3 |
| Plasdone K29/32 | 24.0 |
| Castor oil | 10.7 |
| Cremophor RH 40 | 5.3 |
| Total | 833.3 |
| Solvents | |
| Ethanol 100% | 840.0 |
| Water | 360.0 |

First order release profile (50% release time of 1.7 hours at neutral pH) Coating level = 16%

### Example 6

### Microcapsules M6(a) to M6(d)

Pure amoxicillin granules were coated with a coating composition prepared from ethyl cellulose, PVP, and castor oil (70/23/7), in a solvent system comprising ethanol/acetone in a ratio 60/40 (by weight), to give microcapsules with a coating level of 18%. These, microcapsules had a first order release profile.

A second set of microcapsules (M6(b)) was prepared with the same coating composition, but in which the solvent system was ethanol/water (70/30).

A third set of microcapsules (M6(c)) was prepared by coating pure amoxicillin granules with a coating composition prepared from Aquacoat ECD3 0, triethyl citrate and polyvinylpyrrolidone in the ratio 76/21/3, in an aqueous solvent system, to give microcapsules having a coating level of 15%.

A fourth set of microcapsules (M6(d)) was prepared by coating pure amoxicillin granules with a coating composition prepared from Aquacoat ECD30, dibutyl sebacate and polyvinylpyrrolidone in the ratio 84/13/3, in an aqueous solvent system, to give microcapsules having a coating level of 15%.

### Example 7

### Enteric Coated Microcapsules M7

| | Q (g) |
|---|---|
| Ingredient | |
| Amoxicillin/CLPVP 200-500 μm granulate | 1000.0 |
| Ethocel 7 premium¹ | 45.8 |
| Plasdone K29/32² | 2.1 |
| Dibutyl sebacate | 4.0 |
| Magnesium stearate | 1.1 |
| Solvents | |
| Acetone | 363 |
| Iso-propanol | 242 |

The initially prepared microcapsules (800 mg) were then coated with an enteric coating comprising Eudragit L100 (308.6 g), triethyl citrate (34.3 g) in acetone (2993.2 g) and water (92.6 g).

The microcapsules had an initial coating level of 15%, with a further coating level of 30% for the enteric coating, the diameter of the microcapsules increasing by about 50 μm to about 475 μm.

The dissolution profile was sigmoidal, with a lag time of 1.5 to 2 h, there being no release of amoxicillin at an initial pH of 1.4 over a period of 2 h.

### Example 8

### Measurement of In Vitro Dissolution Profile

The dissolution profile of microcapsules was measured in a dissolution apparatus using a paddle speed of 75 rpm (USP II paddles), at a temperature of 37° C., in a volume of 900 ml and at pH 6.8 (phosphate buffer).

### Example 9

### Suspension Stability Study

The stability of microcapsules in suspension was assessed, as a preliminary step to developing a suspension formulation. Microcapsules (M4) (355 mg) were added to demineralised water (5 ml), to give a suspension which was then stored for ten days at 5° C. The release profile of the granules after 10 days in suspension was then determined and compared to the release profiles of the granules in the initial suspension and when directly measured, using phosphate buffer (900 ml) at pH 6.8, at 75 rpm (USP II paddles).

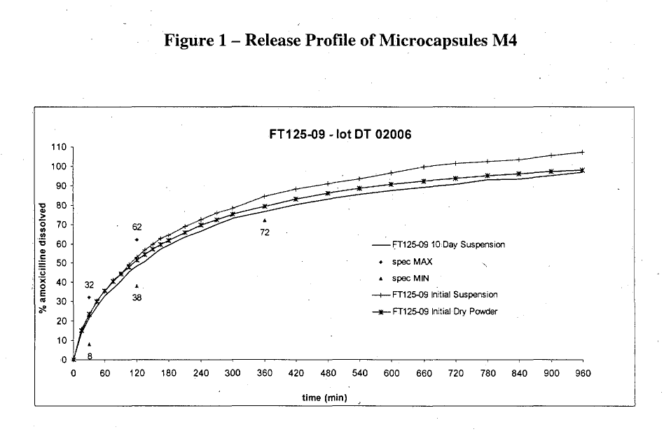

The microcapsules M4 were found to have a similar in vitro release profile after ten days in suspension, compared to the original profiles, as shown in FIG. 1.

In a second study, using the same procedure as before, the stability of a granules in a suspension comprising granules (71%) and immediate release amoxicillin (comprising PVP, 29%) after 7 days storage at 5° C. was investigated. This showed that the microcapsules maintained their in vitro release profile, this effect apparently being assisted by the presence of the immediate release material.

### Examples 10 to 14

### Formulations F1 to F5 Comprising Amoxicillin and Clavulanate

Formulations comprising amoxicillin microcapsules (F1 and F2) and immediate release amoxicillin (F3 to F5) and clavulanate, such that the overall ratio of amoxicillin to clavulanate was 16:1 were prepared for assessment in a pharmacokinetic study using human volunteers.

| Ref | IR % | | SR % | |
|---|---|---|---|---|
| F1 | 0 | granulate | 100 | M1 |
| F2 | 0 | granulate | 100 | M2 |
| F3 | 20 | granulate | 80 | M3 |
| F4 | 40 | | 60 | M4 |
| F5 | 40 | | 60 | M5 |

granulate - amoxicillin plus cross-linked polyvinylpyrrolidone (3%, CLPVP).

For examples, calculations were made on the basis that the theoretical content of amoxicillin pure free base was 84.5% for amoxicillin/CLPVP granulates and 87.1% for pure amoxicillin.

### Example 15

### Pharmacokinetic Study

Formulations F1 to F5 were compared against a modified release tablet formulation of amoxicillin/clavulanate (WO 00/61116), in a human volunteer pharmacokinetic study, using a dosage of 2000/125 mg amoxicillin/clavulanate of each.

The study was an open label, randomised four period partially control-balanced study using 20 subjects, carried out in a manner similar to that described in WO 00/61116, with plasma samples being taken from the subjects over a series of time points, for subsequent analysis for amoxicillin content.

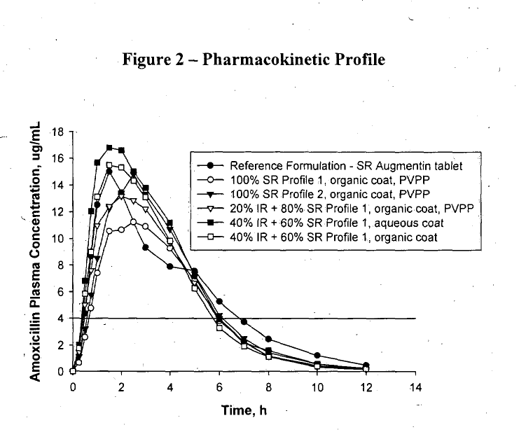

The results of this study are presented in Table 1 and shown graphically in FIG. 2.

### Example 16

### 1000/62.5 and 2000/125 mg Sachets

A. A sachet comprising 2000/125 mg amoxicillin/clavulanate, in which the ratio of amoxicillin in microcapsules to immediate release amoxicillin was 2 to 1, was prepared from the microcapsules described in Example 4 (M4) (corresponding to 1340 mg amoxicillin), amoxicillin/clavulanate as 2:1 granules (corresponding to 250 mg amoxicillin and 125 mg potassium clavulanate¹), amoxicillin as granulates with magnesium stearate (410 mg), silica gel (Syloid A1) (260 mg), aspartame (60 mg) and flavour (strawberry), typically ±10%, ±5%, the weights of the excipients. ¹ amoxicllin (63.3%), and potassium clavulanate (31.7%) in a nominal ratio of 2:1, plus silica gel (3.1%) and cross linked PVP (1.9%) (see WO 98/35672, page 17, Example 5)

B. A sachet comprising 1000/62.5 mg amoxicillin/clavulanate was prepared, comprising half the above quantities.

C. A further sachet comprising 2000/125 mg amoxicillin/clavulanate and in which the ratio of modified release to immediate release amoxicillin was 6:4, was prepared as follows:

| | | mg |
|---|---|---|
| Amoxicillin (microcapsules 250-400 μm) | | 1377 |
| Amoxicillin (250-400 μm) | | 630 |
| 2/1 granule¹ | (Amox/Clav IR) | 471 |
| Amorphous Silica | (Dessicant) | 195 |
| Aspartame | (Sweetener) | 31 |
| Magnesium Stearate | (Lubricant) | 5 |
| Xanthan gum | (Suspending agent) | 44 |
| Strawberry Creme | (Flavour) | 88 |

¹Granule comprising amoxicllin (63.3%), and potassium clavulanate (31.7%) in a nominal ratio of 2:1, plus silica gel (3.1%) and cross linked PVP (1.9%) (see WO 98/35672, page 17, Example 5)
Xanthan gum may be replaced in part by carboxymethylcellulose sodium.

D. Further sachets were prepared in comprising in which the modified release amoxicillin comprised 65, 70 and 87.5% of the total amoxicillin content.

### Example 17

### 400/57 mg/5 ml Suspension

A unit amount of a dry powder formulation for reconstitution to provide to 5 ml of suspensions prepared comprising 400 mg of amoxicillin and 57 mg potassium clavulanate, in which the ratio of amoxicillin in microcapsules to immediate release amoxicillin was 3 to 2, was prepared from the microcapsules described in Example 4 (M4) (corresponding to 240 mg amoxicillin), amoxicillin as granulates with magnesium stearate (160 mg), clavulanate 1:1 blend with silica gel (Syloid A1) (corresponding to 57 mg potassium clavulanate), silica gel (40 mg), xanthan gum (4.4 mg), carboxymethyl cellulose sodium salt (44 mg), sodium benzoate (8 mg), colloidal silica (2.6 mg), aspartame (16 mg), magnesium stearate (1.7 mg) and strawberry flavour (25 mg), typically ±10%, ±5% the weights of the excipients.

The above blend is packed into airtight containers such as glass or plastic bottles and then reconstituted with water prior to first use, to a pre-determined concentration, to provide a multiple dosage product which is stable on storage (at 5° C.) over 10 days.

Similar dry powder formulations may be prepared using the same base and comprising 100/12.5 mg/ml amoxicillin/clavulanate, and 500 mg/5 ml amoxicillin.

### Example 18

### 1000/62.5 mg/5 ml Suspension

| Starting Material | Amount (g) |
|---|---|
| Amoxicillin microcapsules (SR Granules) (250-400 μm) | 27.56 |
| Amoxicillin (IR Granules) | 10.76 |
| Amoxicillin/Clavulanate 2/1 Granule | 9.88 |
| Polyplasdone XL (from 2/1 granule) | 0.30 |
| Magnesium Stearate (from IR granules) | 0.10 |
| Amorphous Silica (from 2/1 granule) | 0.99 |
| Amorphous Silica | 3.33 |
| Sodium Benzoate | 0.34 |
| Magnesium Stearate | 0.07 |
| Aspartame | 0.64 |
| Xanthan Gum | 1.40 |
| Anhydrous Colloidal Silica | 0.10 |
| Strawberry Creme flavour | 1.00 |
| Total | 56.13 |

Notes:
1) 5% overage on potassium clavulanate.
2) This formulation has a re-constitution volume of 200 ml
3) Based on a 60% SR and 40% IR ratio
4) 2/1 granule is described in WO 98/35672 (SmithKline Beecham Laboratoires Pharmaceutique)
5) typically ±10%, ±5% the weights of the excipients.

The excipients used in the previous examples are available as follows:

| Excipient | Manufacturer/ | Specification |
|---|---|---|
| Ethyl cellulose | FMC | USP 25 NF20, page 2546 |
| aqueous | | |
| dispersion | | |
| Aquacoat ECD 30 | | |
| Polyvinyl pyrrolidone | ISP | USP 24 NF19, page 1372 |
| Plasdone K29/32 | | |
| Ethyl cellulose | Dow Chemical | Phar. Eur. 1999, n°822 |
| Ethocel 7 Premium | | |
| Castor oil | Garbit Huilerie | Phar. Eur. 2001, n°51, 1375 |
| Polyoxyl 40 | BASF/Laseron | Phar. Eur. 2002, n°1083, 1633 |
| hydrogenated | | |
| castor oil | | |
| Cremophor RH 40 | | |
| Dibutyl sebacate | Morflex | USP 25 NF20, page 2539 |

### Bioequivalent Formulations

In a further aspect, the present invention also extends to pharmaceutical formulations comprising microcapsules as hereinbefore defined and which are bioequivalent to the formulations of Examples 10 to 18, in terms of both the rate and the extent of absorption, for instance as defined by the US Food and Drug Administration and discussed in the so-called “Orange Book” (Approved Drug Products with Therapeutic Equivalence Evaluations, US Dept of Health and Human Services, 19th edn, 1999). According to this, the PK parameters AUC_{(0-inf)} and Cₘₐₓ should lie within the range 80 to 125% of the corresponding values for the reference formulation. The statistical analysis is carried out using an analysis of variance procedure (ANOVA) and calculating a 90% confidence interval, for each PK parameter, the confidence interval for each parameter lying entirely within the 80 to 125% range.

TABLE 1
Mean (SD) Pharmacokinetic Parameters for Amoxicillin

| | | Cmax | Time > MIC | AUC(0-inf) | Tmax* | T1/2 | AUC(0-t) |
|---|---|---|---|---|---|---|---|
| Regimen | n | (ug/mL) | (h) | (ug · h/mL) | (h) | (h) | (ug · h/mL) |
| Ref | 22 | 16.0 (5.0) | 6.40 (1.59) | 66.2 (17.8) | 1.50 (1.00-5.00) | 1.40 (0.29) | 65.3 (16.9) |
| F1 | 12 | 12.9 (3.9) | 5.10 (0.61) | 54.1 (11.4) | 2.50 (1.50-5.02) | 1.35 (0.20) | 53.7 (11.5) |
| F2 | 13 | 15.7 (4.0) | 5.32 (0.91) | 63.6 (13.6) | 2.50 (1.50-5.00) | 1.34 (0.18) | 63.2 (13.4) |
| F3 | 12 | 14.9 (4.1) | 5.37 (0.95) | 60.8 (17.1) | 2.25 (1.00-5.02) | 1.22 (0.15) | 60.6 (17.0) |
| F4 | 11 | 18.3 (2.3) | 5.82 (1.07) | 73.5 (10.8) | 1.50 (1.00-2.02) | 1.32 (0.15) | 73.0 (10.5) |
| F5 | 13 | 16.1 (4.4) | 5.20 (0.63) | 64.9 (16.7) | 1.53 (1.50-3.00) | 1.35 (0.16) | 64.6 (16.7) |

*Median (range) presented for Tmax

1. Microcapsules for use in a pharmaceutical formulation comprising microparticles of amoxicillin coated with at least one coating film comprising:
(a) at least one film-forming polymer (PI) present in a quantity of 50 to 90% by weight of dry matter of the whole coating composition, and comprising at least one water insoluble cellulose derivative;
(b) at least one nitrogen-containing polymer (P2), present in a quantity of 2 to 25% by weight of dry matter of the whole coating composition, and consisting of at least one polyacrylamide and/or one poly-N-vinylamide and/or one poly-N-vinyl-lactam;
(c) at least one plasticiser present in a quantity of 2 to 25% by weight of dry matter of the whole coating composition, selected from the group consisting of glycerol esters, phtalates, citrates, sebacates, cetylalcohol esters, castor oil and cutin; and
(d) optionally at least one surface-active and/or lubricating agent, present in a quantity of 2 to 20% by weight of dry matter of the whole coating composition, and selected from one or more of anionic surfactants, and/or from nonionic surfactants, and/or from lubricants selected from stearates, stearylfumarates, lauryl sulfate and/or glyceryl behenate;
said microparticles of amoxicillin having a particle size of between 50 and 1000 μm; and
said coating being present in from 5 to 45% by weight of the granule.

2. Microcapsules as claimed in claim 1 in which the film-forming polymer (PI) is present in a quantity of 50 to 80%, more preferably 60 to 80%, by weight of dry matter of the whole coating composition.

3. Microcapsules as claimed in claim 1 in which the film-forming polymer (PI) is ethyl cellulose.

4. Microcapsules as claimed in claim 3 in which the ethyl cellulose is used as an aqueous dispersion in water.

5. Microcapsules as claimed in claim 1 in which the nitrogen-containing polymer (P2) is present in a quantity of 2 to 20%, more preferably 10 to 20%, by weight of dry matter of the whole coating composition.

6. Microcapsules as claimed in claim 1 in which the nitrogen-containing polymer (P2) is polyvinylpyrrolidone.

7. Microcapsules as claimed in claim 1 in which the plasticiser is present in a quantity of 4 to 15%, more preferably 5 to 10%. by weight of dry matter of the whole coating composition.

8. Microcapsules as claimed in claim 1 in which the plasticiser is: dibutyl sebacate, triethyl citrate or castor oil, more preferably castor oil, for use with ethyl cellulose in an organic or aqueous alcohol solvent system solvent; or, a citrate such as triethyl citrate or a sebacate such as dibutyl sebacate, for use with an aqueous dispersion of ethylcellulose.

9. Microcapsules as claimed in claim 1 in which the surface-active and/or lubricating agent is preferably present in a quantity of 2 to 15%, more preferably 2 to 10%, still more preferably 2 to 8%, by weight of dry matter of the whole coating composition.

10. Microcapsules as claimed in claim 1 in which the coating composition comprises ethylcellulose present in from 60 to 80%, polyvinylpyrrolidone present in from 10 to 20%, castor oil present in from 5 to 10%, and polyoxyl 40 hydrogenated castor oil present in from 2 to 8%, by weight of the of dry matter of the whole coating composition.

11. Microcapsules as claimed in claim 10 in which the coating composition, comprises ethyl cellulose (about 70 to 72%), polvinylpyrrolidone (about 16 to 18%), castor oil (about 8%) and polyoxyl 40 hydrogenated castor oil (about 4%), ±5% of each component, by weight of the of dry matter of the whole coating composition.

12. Microcapsules as claimed in claim 1 in which the coating composition is prepared from an aqueous dispersion of ethyl cellulose and comprises ethyl cellulose present in from 60 to 80%, cetyl alcohol present in from 4 to 10%, sodium lauryl sulfate present in from 2 to 5%, polyvinylpyrrolidone present in from 2 to 10%, and dibutyl sebacate present in from 10 to 25%, respectively, by weight of the of dry matter of the whole coating composition.

13. Microcapsules as claimed in claim 12 in which the coating composition comprises ethyl cellulose (about 70 to 72%), polvinylpyrrolidone (about 16 to 18%), castor oil (about 8%) and polyoxyl 40 hydrogenated castor oil (about 4%), ±5% of each component, by weight of the of dry matter of the whole coating composition.

14. Microcapsules as claimed in claim 12 in which the coating composition prepared is from an aqueous dispersion of ethyl cellulose and comprises ethyl cellulose (about 84%), polyvinylpyrrolidone (about 3%), and dibutyl sebacate or triethyl citrate (about 13%), respectively, ±5% of each component, by weight of the three components.

15. Microcapsules as claimed in claim 1 in which the coating represents from 5 to 45%, more preferably from 7 to 20%, advantageously from 10 to 15%, of the weight of the microcapsules.

16. Microcapsules as claimed in claim 1 in which have a second coating layer which is an enteric coating.

17. Microcapsules as claimed in claim 1 in which the particle size of the amoxicillin microparticles is between 100 and 750 μm, more preferably, between 100 and 500 μm, advantageously between 200 and 400 μm, more advantageously between 250 and 400 μm.

18. Microcapsules as claimed in claim 1 which comprise amoxicillin in an amount of between 55 and 95%, more preferably between 75 and 95%, advantageously between 85 and 95%, by weight of the microcapsule.

19. Microcapsules as claimed in claim 1 which release between 10 and 30% amoxicillin after 0.5 h, between 40 and 60% after 2 h and at least 70% after 6 h (measured at pH 6.8).

20. A process for preparing microcapsules of amoxicillin as defined in any one of the preceding claims which process comprises applying in a film coating apparatus a coating composition prepared by admixing the components of the film coating in a solvent system to microparticles of amoxicillin and thereafter drying the microcapsules thus obtained, and optionally, and if necessary, admixing these microcapsules with at least one anti-agglomerating agent.

21. A process as for preparing microcapsules as claimed in claim 20 in which the solvent is an aqueous alcohol.

22. A process as for preparing microcapsules as claimed in claim 20 in which the solvent is water.

23. Microcapsules as claimed in claim 1 obtainable by applying a coating composition prepared by admixing the components of the film coating in an aqueous solvent system to microparticles of amoxicillin and thereafter drying the microcapsules thus obtained.

24. A modified release pharmaceutical formulation which comprises amoxicillin, and optionally potassium clavulanate such that the ratio of amoxicillin to clavulanate (if present) is from 2:1 to 30:1, and in which from 30 to 100% of the amoxicillin content is present as microcapsules of amoxicillin, as defined in any of claims 1 to 21 or 25, to provide a slow release phase and from 0 to 70% of the amoxicillin content is present in an immediate release form, to provide an immediate release phase, further admixed with pharmaceutically acceptable excipients.

25. A modified release pharmaceutical formulation as claimed in claim 24 in which the ratio of amoxicillin to clavulanate is from 2:1 to 20:1, more preferably 6:1 to 20:1, more preferably 12:1 to 20:1, most preferably 14:1 to 16:1.

26. A modified release pharmaceutical formulation as claimed in claim 24 in which from 40 to 100%, more preferably from 50 to 90%, of the amoxicillin content is present as microcapsules of amoxicillin.

27. A modified release pharmaceutical formulation as claimed in claim 26 in which the Area Under the Curve (AUC) value is substantially similar to, for instance at least 80%, preferably at least 90%, of that of the corresponding dosage of amoxicillin taken as a conventional (immediate release) formulation, over the same dosage period.

28. A modified release pharmaceutical formulation as claimed in claim 24 which is provided as a single dose sachet.

29. A modified release pharmaceutical formulation as claimed in claim 28 which comprises from 250 to 3000 mg of amoxicillin, for instance 250, 500, 600, 800, 1000, 1500, 2000, 2500, 3000 mg amoxicillin.

30. A modified release pharmaceutical formulation as claimed in claim 28 which comprises from 2000 mg amoxicillin and 125 mg of clavulanate.

31. A modified release pharmaceutical formulation as claimed in claim 24 which is provided as a dry powder formulations for paediatric use for reconstitution into aqueous suspension.

32. A modified release pharmaceutical formulation as claimed in claim 31 which comprises from 200 to 1200 mg of amoxicillin per unit amount of formulation, and optionally from 25 to 80 mg of clavulanate per unit amount of formulation, such that the ratio of amoxicillin to clavulanate is in the range 2:1 to 30:1.

33. A modified release pharmaceutical formulation as claimed in claim 24 which provides a mean plasma concentration of amoxicillin of 4 μg/mL for at least 4.6 h, preferably at least 4.8 h, more preferably at least 5.5 h, typically between 5.5 and 6.5 h.

34. A modified release pharmaceutical formulation as claimed in claim 24 which provides mean maximum plasma concentration (Cₘₐₓ) of amoxicillin which is at least 12 μg/mL, preferably at least 14 μg/mL, most preferably at least 16 μg/mL, typically from 16 to 20 μg/mL.

35. A modified release pharmaceutical formulation comprising microcapsules as defined in claim 1 and which is bioequivalent to any one of the formulations hereinbefore exemplified in Examples 10 to 14 and Examples 16 and 17.

36. The use of amoxicillin and optionally clavulanate in the manufacture of a medicament, as defined in claim 24, for treating bacterial infections, in particular in adult patients, at intervals of about 12 h.

37. A method of treating bacterial infection in a paediatric patient which method comprises administering to a patient in need thereof a dosage of from 80 to 180 mg/kg/day of amoxicillin, preferably from 120 to 160, more preferably about 150 mg/kg/day and, optionally, from 6 to 11 mg/kg/day, in divided dosages every 12 h, from a modified release formulation.
